(19) United States
(12) Patent Application Publication
(10) Pub. No.: US 2024/0041625 A1
(43) Pub. Date: Feb. 8, 2024

(54) MULTIPLE STENT DELIVERY SYSTEM

(71) Applicant: KONINKLIJKE PHILIPS N.V., EINDHOVEN (NL)

(72) Inventors: Andrew Scherer, MAPLE GROVE, MN (US); Scott Robert Wilson, MAPLE GROVE, MN (US); Cedar Decker, ALBERTVILLE, MN (US); Jake Mertens, MAPLE GROVE, MN (US)

(21) Appl. No.: 18/266,025

(22) PCT Filed: Dec. 13, 2021
(86) PCT No.: PCT/EP2021/085363

§ 371 Date: Jun. 8, 2023

Related U.S. Application Data
(60) Provisional application No. 63/127,239, filed on Dec. 18, 2020.

(51) Int. Cl. A61F 2/954; A61F 2/966; A61F 2/95; G16H 20/40

(52) U.S. Cl. CPC A61F 2/954; A61F 2/966; A61F 2/9517; G16H 20/40; A61F 2002/826

(57) ABSTRACT

A stent delivery system (100) includes a first outer sheath (111) configured to surround a first stent (110); and a second outer sheath (121) configured to surround a second stent (120).

### BACKGROUND

A stent is a tube inserted into a blocked passageway to keep the passageway open. Expandable metal stents may be delivered intravascularly to treat intravascular disease. Expandable metal stents may also be used to treat larger regions of anatomy such as the esophagus, biliary, and pulmonary systems. A physician may place a single stent to address a vessel narrowing or restriction. A physician may also deploy two adjoining stents to equally support two pathways at an arterial bifurcation or venous confluence. Arterial bifurcation is a term that describes branching or forking in the vascular system, and venous confluence is a term that describes when two veins converge in the vascular system. Currently, a physician may switch from operating one delivery mechanism for delivering one stent to another delivery mechanism for deploying the other stent, in an attempt to keep the deployments balanced. Physicians may need to access vasculature on both the left side and the right side.

### SUMMARY

According to an aspect of the present disclosure, a stent delivery system includes a first outer sheath configured to surround a first stent; and a second outer sheath configured to surround a second stent.

According to another aspect of the present disclosure, a catheter includes a first outer sheath configured to surround a first stent; and a second outer sheath around the second stent. In the catheter, the first stent is offset from the second stent along an axis of the catheter when the first stent and the second stent are assembled with the catheter.

According to yet another aspect of the present disclosure, a method of deploying stents from a stent delivery system includes positioning the stent delivery system at a first disease site in an anatomical pathway and deploying a first stent from the stent delivery system at the first disease site. A first outer sheath is retracted from around the first stent while deploying the first stent from the stent delivery system. The method also includes positioning the stent delivery system at a second disease site in the anatomical pathway and deploying a second stent from the stent delivery system at the second disease site. A second outer sheath is retracted from around the second stent while deploying the second stent from the stent delivery system.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 illustrates a deployment of stents via a multiple stent delivery system, in accordance with a representative embodiment.

FIG. 2 illustrates another deployment of stents via a multiple stent delivery system, in accordance with a representative embodiment.

FIG. 3 illustrates another deployment of stents via a multiple stent delivery system, in accordance with a representative embodiment.

FIG. 4 illustrates a multiple stent delivery system, in accordance with a representative embodiment.

FIG. 5 illustrates another multiple stent delivery system, in accordance with a representative embodiment.

FIG. 6 illustrates another multiple stent delivery system, in accordance with a representative embodiment.

FIG. 7 illustrates another deployment of stents via a multiple stent delivery system, in accordance with a representative embodiment.

FIG. 8 illustrates another multiple stent delivery system, in accordance with a representative embodiment.

FIG. 9 illustrates another multiple stent delivery system, in accordance with a representative embodiment.

FIG. 10 illustrates another multiple stent delivery system, in accordance with a representative embodiment.

FIG. 11 illustrates a method of operation for a multiple stent delivery system, in accordance with a representative embodiment.

FIG. 12 illustrates a computer system on which a method of operation for a multiple stent delivery system is implemented, in accordance with a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, a multiple stent delivery system is configured to deploy multiple stents at the same time from a single access site. The multiple stent delivery system provides for placing multiple stents using a single dedicated device, and requiring only one vascular access site.

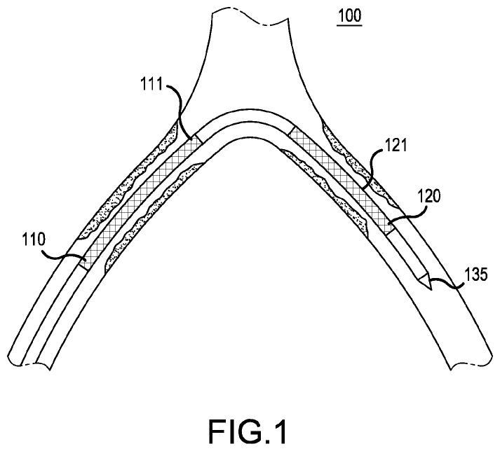

FIG. 1 illustrates a deployment of stents via a multiple stent delivery system, in accordance with a representative embodiment.

In FIG. 1, a multiple stent delivery system 100 includes a catheter 101. The catheter 101 in FIG. 1 comprises a first outer sheath 111 configured to surround a first stent 110, a second outer sheath 121 configured to surround a second stent 120, and an endpoint 135. In other words, a multiple stent delivery system 100 may include, as an integrated unit of assembled components when the first stent 110 and the second stent 120 are assembled with the multiple stent delivery system 100, the first stent 110, the first outer sheath 111, the second stent 120, and the second outer sheath 121. The multiple stent delivery system 100 may be assembled so that an axis of the multiple stent delivery system 100 is a central axis that starts at the endpoint of 135, and that runs through the second stent 120 and the second outer sheath 121, and through the first stent 110 and the first outer sheath 111. The multiple stent delivery system 100 may be flexible so that the multiple stent delivery system 100 is flexibly adjusted to pass through non-linear structures in anatomy as shown in FIG. 1. Also in FIG. 1, shaded or hatched portions immediately adjacent to outer edged of the branched anatomy indicate disease sites where stents will be placed. The multiple stent delivery system 100 may be assembled with the first stent 110, the first outer sheath 111, the second stent 120 and the second outer sheath 121. The catheter 101 may include the first stent 110, the first outer sheath 111, the second stent 120 and the second outer sheath 121 until the first stent 110 and the second stent 120 are deployed. The first stent 110 and the second stent 120 may be deployed via a single vascular access site and via a single catheter insertion.

In FIG. 1, the catheter 101 that comprises the first stent 110 and the second stent 120 deploys both stents via a single insertion of the catheter 101. As explained herein, the first stent 110 and the second stent 120 may be aligned in the catheter 101, so that deployment may involve retracting the first outer sheath 111, deploying the first stent 110, retracting the second outer sheath 121, and then deploying the second stent 120. As an example reflected in FIG. 1, vascular disease in the iliac region is shown on both the left side and the right side. The catheter 101 is a single catheter and is used in a single catheter insertion on the lower left side in FIG. 1. The catheter 101 is positioned so that the second stent 120 is deployed on the right side and the first stent 110 is deployed on the left side. The second stent 120 is deployed within the right side disease site D2, and the first stent 110 is deployed within the left side disease site D1.

In FIG. 1, the first stent 110 is deployed by retracting the first outer sheath. While the first stent is deployed, the second stent 120 remains within the second outer sheath.

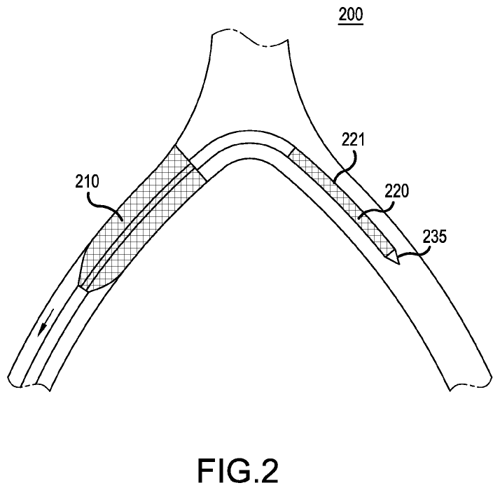

FIG. 2 illustrates another deployment of stents via a multiple stent delivery system, in accordance with a representative embodiment.

In FIG. 2, a multiple stent delivery system 200 includes a catheter 201. The catheter 201 in FIG. 2 comprises a first stent 210, a second stent 220, a second outer sheath 221, and an endpoint 235. In FIG. 2, there is not a first outer sheath corresponding to the first outer sheath 111 in FIG. 1 because the first outer sheath is retracted to allow the first stent 210 to deploy. The first stent 210 and the second stent 220 may each be self-expanding so that they each expand to their expected dimensions as their outer sheaths are retracted. The multiple stent delivery system 200 may be assembled with the first stent 210, the first outer sheath 211, the second stent 220 and the second outer sheath 221. The catheter 201 may include the first stent 210, the first outer sheath 211, the second stent 220 and the second outer sheath 221 until the first stent 210 and the second stent 220 are deployed. The first stent 210 and the second stent 220 may be deployed via a single vascular access site and via a single catheter insertion.

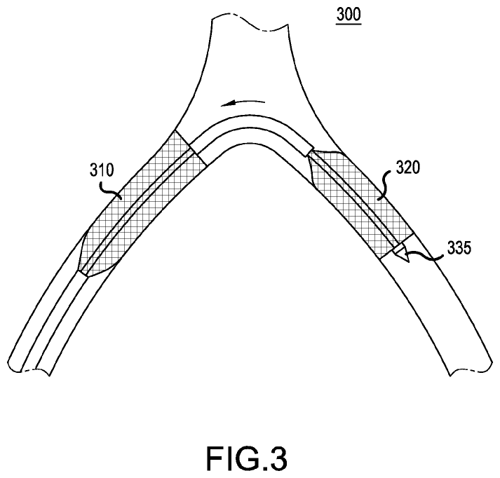

FIG. 3 illustrates another deployment of stents via a multiple stent delivery system, in accordance with a representative embodiment.

In FIG. 3, a multiple stent delivery system 300 includes a catheter 301. The catheter 301 in FIG. 3 comprises a first stent 310, a second stent 320 and an endpoint 335. In FIG. 3, there is not a first outer sheath corresponding to the first outer sheath 111 in FIG. 1 because the first outer sheath is retracted to allow the first stent 310 to deploy. In FIG. 3, there is not a second outer sheath corresponding to the second outer sheath 121 in FIG. 1 or to the second outer sheath 221 in FIG. 2 because the second outer sheath is retracted to allow the second stent 320 to deploy. The multiple stent delivery system 300 may be assembled with the first stent 310, the first outer sheath 311, the second stent 320 and the second outer sheath 321. The catheter 301 may include the first stent 310, the first outer sheath 311, the second stent 320 and the second outer sheath 321 until the first stent 310 and the second stent 320 are deployed. The first stent 310 and the second stent 320 may be deployed via a single vascular access site and via a single catheter insertion.

In FIG. 3, the first stent 310 has been deployed, and the catheter 301 is then moved to center the second stent 320 over the disease on the right side. The second stent 320 is deployed by retracting the second outer sheath 321.

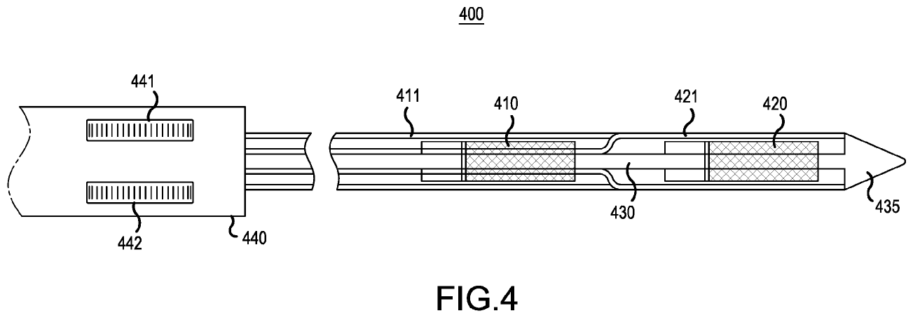

FIG. 4 illustrates a multiple stent delivery system, in accordance with a representative embodiment.

In FIG. 4, a multiple stent delivery system 400 includes a handle 440 and a catheter 401. The handle 440 includes a first thumbwheel 441 and a second thumbwheel 442. The catheter 401 in FIG. 4 comprises a first stent 410, a first outer sheath 411, a second stent 420, a second outer sheath 421, an inner support shaft 430 and an endpoint 435. The first thumbwheel 441 is a drive system used to retract the first outer sheath 411. The second thumbwheel 442 is a drive system used to retract the second outer sheath 421. The multiple stent delivery system 400 may be assembled with the first stent 410, the first outer sheath 411, the second stent 420 and the second outer sheath 421. The catheter 401 may include the first stent 410, the first outer sheath 411, the second stent 420 and the second outer sheath 421 until the first stent 410 and the second stent 420 are deployed. The first stent 410 and the second stent 420 may be deployed via a single vascular access site and via a single catheter insertion.

As shown in FIG. 4, the second outer sheath 421 encloses the second stent 420, but is also enclosed within the first stent 410 around the inner support shaft 430. When the second outer sheath 421 is retracted, the second outer sheath 421 is retracted from around the second stent 420 and within the first stent 410.

The handle 440 is a handle assembly containing the first thumbwheel 441 and the second thumbwheel 442 to control corresponding outer sheaths. In the embodiment of FIG. 4, the first stent 410 and the second stent 420 are each constrained by an individual outer sheath, and this allows one stent to be positioned and deployed while the other stent is still constrained. Once the first stent 410 is deployed, the second stent 420 can be positioned and deployed. The first stent 410 may be crimped over the second outer sheath 421 when the multiple stent delivery system 400 is assembled. For example, the second outer sheath 421 may be navigated through the first stent 410. In use, the second outer sheath 421 may be retracted through the first stent 410 when the second stent 420 is deployed and the second outer sheath 421 is retracted. The second outer sheath 421 is at full diameter at the distal section over the length of the second stent 420, and has a reduced diameter through the inside of the first stent 410 and through the remaining length of the catheter 401. The first outer sheath 411 is controlled by the first thumbwheel 441, and the second outer sheath 421 is controlled by the second thumbwheel 442, whereby rotation of each thumbwheel results in linear retraction of the corresponding outer sheath. The inner support shaft 430 remains stationary throughout the deployment process.

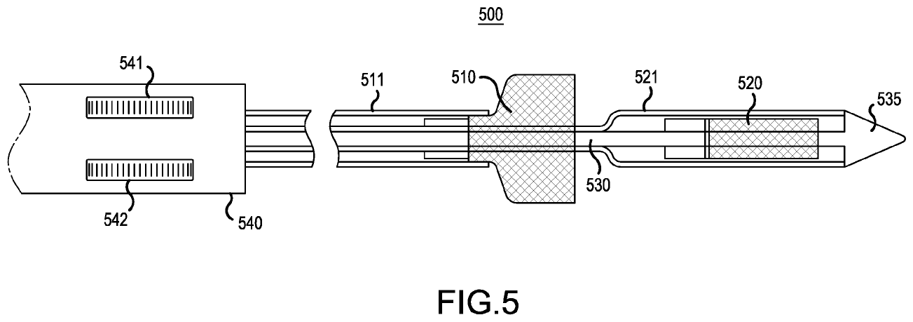

FIG. 5 illustrates another multiple stent delivery system, in accordance with a representative embodiment.

In FIG. 5, a multiple stent delivery system 500 includes a handle 540 with a first thumbwheel 541 and a second thumbwheel 542. The multiple stent delivery system 500 also includes a first stent 510, a first outer sheath 511, a second stent 520, a second outer sheath 521, an inner support shaft 530 and an endpoint 535. The first thumbwheel 541 is a drive system used to retract the first outer sheath 511. The second thumbwheel 542 is a drive system used to retract the second outer sheath 521. The multiple stent delivery system 500 may be assembled with the first stent 510, the first outer sheath 511, the second stent 520 and the second outer sheath 521. A catheter (not shown in FIG. 5) may include the first stent 510, the first outer sheath 511, the second stent 520 and the second outer sheath 521 until the first stent 510 and the second stent 520 are deployed. The first stent 510 and the second stent 520 may be deployed via a single vascular access site and via a single catheter insertion.

As shown in FIG. 5, the first outer sheath 511 has been withdrawn partially to allow the first stent 510 to deploy. The second outer sheath 521 continues to enclose the second stent 520, and is still enclosed within the first stent 510 around the inner support shaft 530.

To deploy the first stent 510 and the second stent 520 in FIG. 5, the first stent 510 may be deployed first. Rotation of the first thumbwheel 541 retracts the first outer sheath 511, and allowing the first stent 510 to deploy into the vessel as shown in FIG. 5. The first stent 510 and the second stent 520 may each be self expanding, so that once constraint is removed, each stent automatically expands into an expected form with expected dimensions such as diameter.

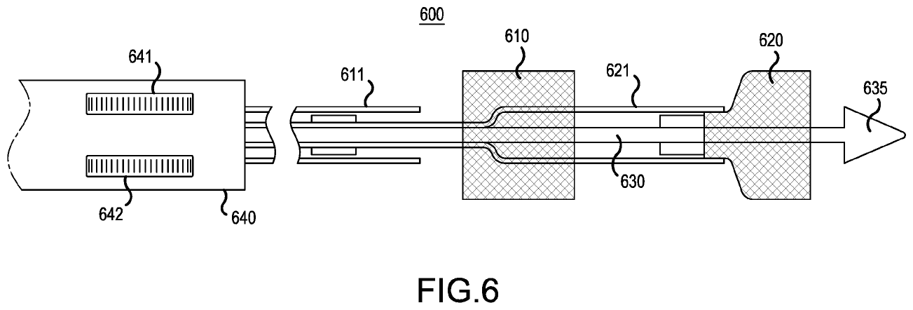

FIG. 6 illustrates another multiple stent delivery system, in accordance with a representative embodiment.

In FIG. 6, a multiple stent delivery system 600 includes a handle 640 with a first thumbwheel 641 and a second thumbwheel 642. The multiple stent delivery system 600 also includes a first stent 610, a first outer sheath 611, a second stent 620, a second outer sheath 621, an inner support shaft 630 and an endpoint 635. The first thumbwheel 641 is a drive system used to retract the first outer sheath 611. The second thumbwheel 642 is a drive system used to retract the second outer sheath 621. The multiple stent delivery system 600 may be assembled with the first stent 610, the first outer sheath 611, the second stent 620 and the second outer sheath 621. A catheter (not shown in FIG. 6) may include the first stent 610, the first outer sheath 611, the second stent 620 and the second outer sheath 621 until the first stent 610 and the second stent 620 are deployed. The first stent 610 and the second stent 620 may be deployed via a single vascular access site and via a single catheter insertion.

As shown in FIG. 6, the first outer sheath 611 has been withdrawn partially to allow the first stent 610 to deploy. The second outer sheath 621 has also been withdrawn partially to allow the second stent 620 to deploy, but is still enclosed within the first stent 610 around the inner support shaft 630. Once the first stent 610 is deployed, the multiple stent delivery system 600 can be positioned such that the second stent 620 is located in a desired position. Rotating the second thumbwheel 642 retracts the second outer sheath 621, allowing the second stent 620 to deploy into the vessel. The first stent 610 and the second stent 620 may each be self expanding. Once the first stent 610 and the second stent 620 are deployed, the remaining elements of the multiple stent delivery system 600 may be withdrawn.

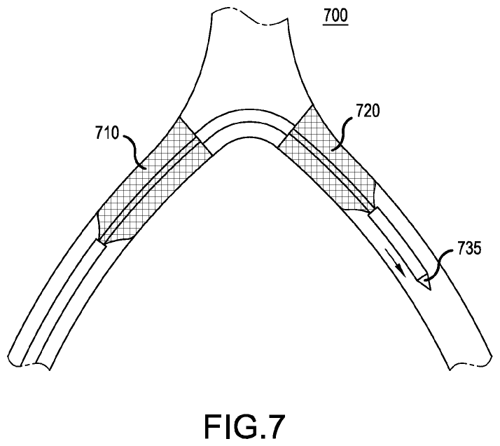

FIG. 7 illustrates another deployment of stents via a multiple stent delivery system, in accordance with a representative embodiment.

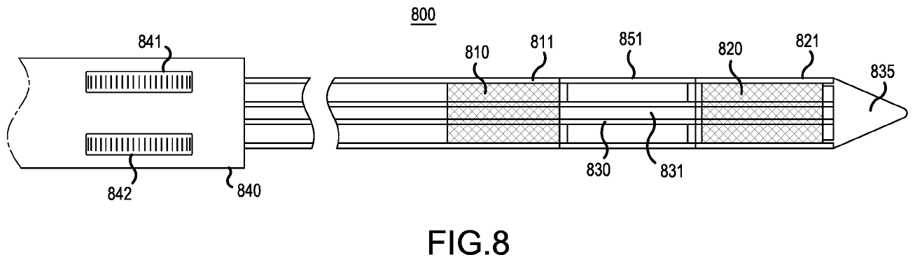

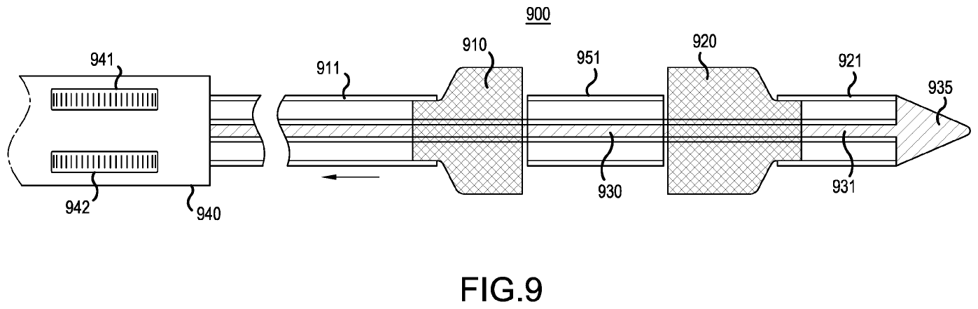

In FIG. 7, a multiple stent delivery system 700 includes a catheter 701. The catheter 701 in FIG. 7 comprises a first stent 710, a second stent 720 and an endpoint 735. Although not shown, the multiple stent delivery system 700 may also include a first outer sheath (not shown) and a second outer sheath (not shown), so that the multiple stent delivery system 100 may include, as an integrated unit of assembled components, the first stent 710, the first outer sheath (not shown), the second stent 720, and the second outer sheath (not shown) when the first stent 710 and the second stent 720 are assembled with the catheter 701. Before the first stent 710 and the second stent 720 are assembled with the catheter 701, the catheter 701 may include the first outer sheath (not shown) configured to surround the first stent 710 and the second outer sheath (not shown) configured to surround the second stent 720. The multiple stent delivery system 700 may be assembled so that an axis of the multiple stent delivery system 700 is a central axis that starts at the endpoint of 735, and that runs through the second stent 720 and the second outer sheath (not shown), and through the first stent 710 and the first outer sheath (not shown). The multiple stent delivery system 700 may be flexible so that the multiple stent delivery system 700 may be flexibly adjusted to pass through non-linear structures in anatomy as shown in FIG. 7. The embodiment of FIG. 7, FIG. 8 and FIG. 9 differs from the embodiment of FIG. 1, FIG. 2, FIG. 3, FIG. 4, FIG. 5 and FIG. 6. As shown in FIG. 7, the first stent 710 deploys in the same direction as the first stent 210 in FIG. 2 and the first stent 310 in FIG. 3. However, the second stent 720 in FIG. 7 deploys in the opposite direction compared to the second stent 320 in FIG. 3. The multiple stent delivery system 700 may be assembled with the first stent 710, the first outer sheath 711, the second stent 720 and the second outer sheath 721. The catheter 701 may include the first stent 710, the first outer sheath 711, the second stent 720 and the second outer sheath 721 until the first stent 710 and the second stent 720 are deployed. The first stent 710 and the second stent 720 may be deployed via a single vascular access site and via a single catheter insertion.

FIG. 8 illustrates another multiple stent delivery system, in accordance with a representative embodiment.

In FIG. 8, a multiple stent delivery system 800 includes a handle 840 and a catheter 801. The handle 840 includes a first thumbwheel 841 and a second thumbwheel 842. The catheter 801 in FIG. 8 comprises a first stent 810, a first outer sheath 811, a second stent 820, a second outer sheath 821, a third outer sheath 851, a first inner support shaft 830, a second inner support shaft 831 and an endpoint 835. The first thumbwheel 841 is a drive system used to retract the first outer sheath 811. The second thumbwheel 842 is a drive system used to retract the second outer sheath 821, but in the opposite direction compared to the second outer sheath 421 in FIG. 4, the second outer sheath 521 in FIG. 5 and the second outer sheath 621 in FIG. 6. The multiple stent delivery system 800 may be assembled with the first stent 810, the first outer sheath 811, the second stent 820 and the second outer sheath 821. The catheter 801 may include the first stent 810, the first outer sheath 811, the second stent 820 and the second outer sheath 821 until the first stent 810 and the second stent 820 are deployed. The first stent 810 and the second stent 820 may be deployed via a single vascular access site and via a single catheter insertion.

In the embodiment of FIG. 7, FIG. 8 and FIG. 9, two stents can be deployed such that the deployments of both stents start at the confluence. This allows improved placement accuracy in the important region where both vessels come together. The embodiment of FIG. 7, FIG. 8 and FIG. 9, includes two inner shafts. As explained below with respect to FIG. 8, the first inner support shaft 830 provides support for the first stent 810 and second stent 820 which are each crimped. That is, the first stent 810 and the second stent 820 are constrained until deployment. The first stent 810 is constrained by the first outer sheath 811 and the second stent 820 is constrained by the second outer sheath 821. The first inner support shaft 830 remains stationary throughout the deployment process. The second inner support shaft 831 is provided for the second outer sheath 821 and runs through the inside diameter of the first inner support shaft 830, and is connected to the second outer sheath 821 at the distal tip. The embodiment of FIG. 7, FIG. 8 and FIG. 9 also includes a third outer sheath 851, which is a stationary sheath to cover the section between the first stent 810 and the second stent 820.

As shown in FIG. 8, the second outer sheath 821 encloses the second stent 820, but is not enclosed within the first stent 810 around the first inner support shaft 830. That is, the second outer sheath 821 is differently configured compared to the second outer sheath 421 in FIG. 4, the second outer sheath 521 in FIG. 5 and the second outer sheath 621 in FIG. 6. When the second outer sheath 821 is retracted, the second outer sheath 821 is retracted from around the second stent 820 to the right in FIG. 8, and is advanced by the second inner support shaft 831 driven by the second thumbwheel 842. Accordingly, in FIG. 8, instead of having the first stent 810 and the second stent 820 deploy in the same direction, the first stent 810 is deployed by pulling back, and the second stent 820 is deployed by pushing forward. The multiple stent delivery system 800 may be assembled with the first stent 810 and the second stent arranged in a catheter oriented in opposite directions, so that they are deployed in the opposite directions in use.

FIG. 9 illustrates another multiple stent delivery system, in accordance with a representative embodiment.

In FIG. 9, a multiple stent delivery system 900 includes a handle 940. The handle 940 includes a first thumbwheel 941 and a second thumbwheel 942. The multiple stent delivery system 900 also includes a first stent 910, a first outer sheath 911, a second stent 920, a second outer sheath 921, a third outer sheath 951, a first inner support shaft 930, a second inner support shaft 931 and an endpoint 935. The first thumbwheel 941 is a drive system used to retract the first outer sheath 911. The second thumbwheel 942 is a drive system used to retract the second outer sheath 921, but in the opposite direction compared to the second outer sheath 421 in FIG. 4, the second outer sheath 521 in FIG. 5 and the second outer sheath 621 in FIG. 6. The multiple stent delivery system 900 may be assembled with the first stent 910, the first outer sheath 911, the second stent 920 and the second outer sheath 921. A catheter (not shown in FIG. 9) may include the first stent 910, the first outer sheath 911, the second stent 920 and the second outer sheath 921 until the first stent 910 and the second stent 920 are deployed. The first stent 910 and the second stent 920 may be deployed via a single vascular access site and via a single catheter insertion.

To deploy the first stent 910 and the second stent 920, the multiple stent delivery system 900 is positioned in the vasculature, with the first stent 910 placed in the desired location. The first thumbwheel 941 is rotated, causing the first outer sheath 911 to retract, allowing the first stent 910 to deploy into the vessel. The first stent 910 and the second stent 920 may each be self expanding. The multiple stent delivery system 900 can then be moved such that the second stent 920 is located in the desired location. The second thumbwheel 942 is rotated, causing the second inner support shaft 931 and the second outer sheath 921 to move distally, allowing the second stent 920 to self-expand and deploy into the vessel with the proximal end first as shown in FIG. 9.

As shown in FIG. 9, the second outer sheath 921 encloses the second stent 920, but is not enclosed within the first stent 910 around the first inner support shaft 930. That is, the second outer sheath 921 is differently configured compared to the second outer sheath 421 in FIG. 4, the second outer sheath 521 in FIG. 5 and the second outer sheath 621 in FIG. 6. When the second outer sheath 921 is retracted, the second outer sheath 921 is retracted from around the second stent 920 to the right in FIG. 9, and is advanced by the second inner support shaft 931 driven by the second thumbwheel 942.

As shown in FIG. 9, instead of having the first stent 910 and the second stent 920 deploy in the same direction, the first stent 910 is deployed by pulling back, and the second stent 920 is deployed by pushing forward.

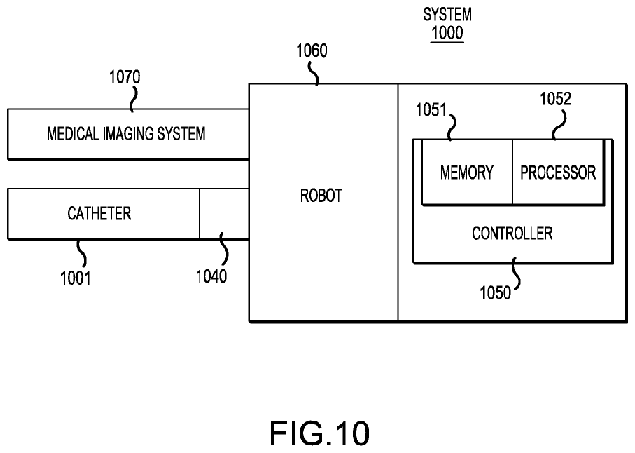

FIG. 10 illustrates another multiple stent delivery system, in accordance with a representative embodiment.

As shown, the multiple stent delivery system 1000 includes a catheter 1001, a handle 1040, a controller 1050, a robot 1060, and a medical imaging system 1070. The catheter 1001 and the handle 1040 may comprise the multiple stent delivery systems from embodiments based on FIGS. 1-6 and based on FIG. 7-9. Additionally, the medical imaging system 1070 may be, as non-limiting examples, an X-ray imaging system or an ultrasound imaging system, and may be used for imaging in interventional medical procedures in embodiments based on FIGS. 1-6 and based on FIG. 7-9.

The robot 1060 may control the use of the multiple stent delivery systems described herein. The robot 1060 may be configured to drive deployment of the first stent and the second stent in each embodiment by deploying the first stent, then retracting the first outer sheath while the second stent remains within the second outer sheath, then deploying the second stent, and then retracting the second outer sheath. In the embodiment of FIGS. 1-6, the robot 1060 may retract both sheaths in the same direction, i.e., towards the handle. In the embodiment of FIGS. 7-9, the robot 1060 may retract the first sheath towards the handle and the second outer sheath towards the distal end of the catheter. The robot 1060 may operate in accordance with instructions executed by the controller 1050, and the controller 1050 may execute instructions based on automated image analysis based on images from the medical imaging system 1070. The controller 1050 includes a memory 1051 that stores instructions and a processor 1052 that executes the instructions. When executed by the processor 1052, the instructions may cause the robot 1060 to implement some or all appropriate aspects of methods and functionality described herein.

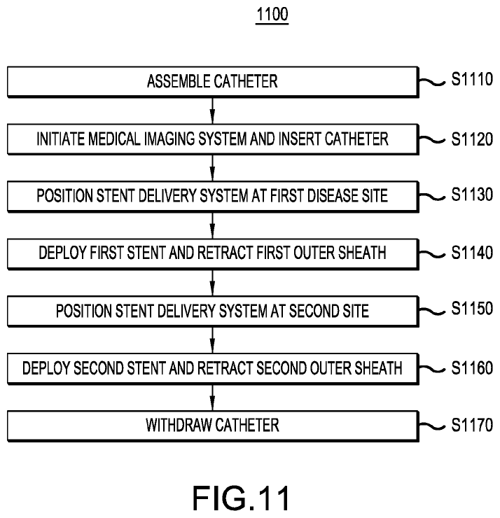

FIG. 11 illustrates a method of operation for a multiple stent delivery system, in accordance with a representative embodiment.

At S1110, the method of FIG. 11 includes assembling a catheter. The catheter may be a catheter used in any of the embodiments described herein, and may be assembled to include the stents, outer sheaths, and inner supports described herein.

At S1120, the method of FIG. 11 includes initiating medical imaging and inserting the catheter. The medical imaging may be implemented by the medical imaging system 1070, and may be performed throughout processes when catheters are inserted into patients during an interventional medical procedure. The catheter may be inserted by a physician or by the robot 1060.

At S1130, the stent delivery system may be positioned at a first disease site. More particularly, a first stent may be positioned by the stent delivery system at the first disease site. The positioning may be based in part on images from the medical imaging system 1070 in FIG. and may be performed by a physician or by the robot 1060.

At S1140, the first stent may be deployed and the first outer sheath is retracted. The first outer sheath may be retracted before the first stent is deployed, or for a self-expanding first stent the first stent may be deployed as the first outer sheath is retracted.

At S1150, the stent delivery system is positioned at a second disease site. More particularly, a second stent may be positioned by the stent delivery system at the second disease site. The positioning at S1150 may be based in part on images from the medical imaging system 1070 in FIG. 10, and may be performed by a physician or by the robot 1060.

At S1160, the second stent may be deployed and the second outer sheath is retracted. The second outer sheath may be retracted before the second stent is deployed, or for a self-expanding second stent the second stent may be deployed as the second outer sheath is retracted.

At S1170, the catheter is withdrawn from the patient. The catheter may be withdrawn by a physician or by the robot 1060.

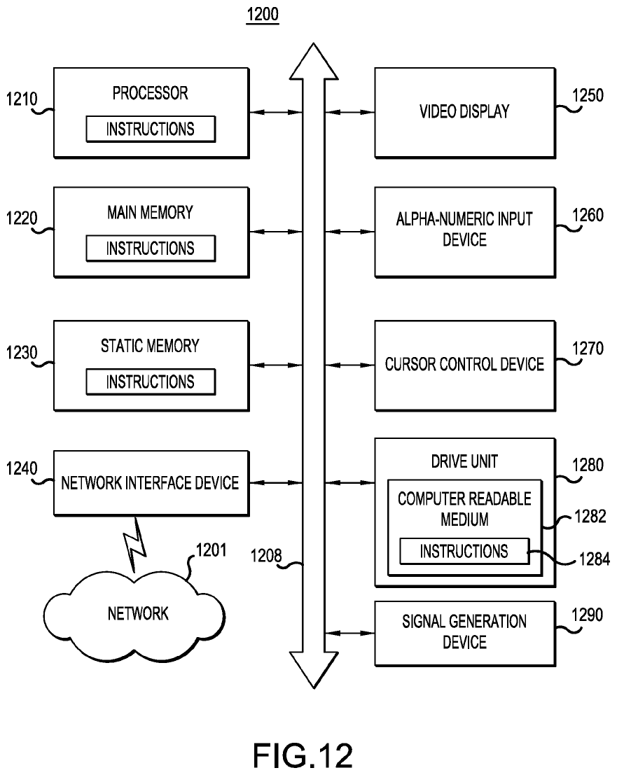

FIG. 12 illustrates a computer system on which a method of operation for a multiple stent delivery system is implemented, in accordance with a representative embodiment.

The computer system 1200 of FIG. 12 shows a complete set of components for a communications device or a computer device. However, a “controller” as described herein may be implemented with less than the set of components of FIG. 12, such as by a memory and processor combination as shown for the controller 1050 in FIG. 10. The computer system 1200 may include some or all elements of a robot 1060 or a controller 1050 used in the multiple stent delivery system herein, although any such apparatus may not necessarily include one or more of the elements described for the computer system 1200 and may include other elements not described.

Referring to FIG. 12, the computer system 1200 includes a set of software instructions that can be executed to cause the computer system 1200 to perform any of the methods or computer-based functions disclosed herein. The computer system 1200 may operate as a standalone device or may be connected, for example, using a network 1201, to other computer systems or peripheral devices. In embodiments, a computer system 1200 performs logical processing based on digital signals received via an analog-to-digital converter.

In a networked deployment, the computer system 1200 operates in the capacity of a server or as a client user computer in a server-client user network environment, or as a peer computer system in a peer-to-peer (or distributed) network environment. The computer system 1200 can also be implemented as or incorporated into various devices, such as the robot 1060 in FIG. 10 or a workstation or computer that includes the controller 1050 in FIG. 10, or any other machine capable of executing a set of software instructions (sequential or otherwise) that specify actions to be taken by that machine. The computer system 1200 can be incorporated as or in a device that in turn is in an integrated system that includes additional devices. In an embodiment, the computer system 1200 can be implemented using electronic devices that provide voice, video or data communication. Further, while the computer system 1200 is illustrated in the singular, the term “system” shall also be taken to include any collection of systems or sub-systems that individually or jointly execute a set, or multiple sets, of software instructions to perform one or more computer functions.

As illustrated in FIG. 12, the computer system 1200 includes a processor 1210. The processor 1210 may be considered a representative example of the processor 1052 of the controller 1050 in FIG. 10 and executes instructions to implement some or all aspects of methods and processes described herein. The processor 1210 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 1210 is an article of manufacture and/or a machine component. The processor 1210 is configured to execute software instructions to perform functions as described in the various embodiments herein. The processor 1210 may be a general-purpose processor or may be part of an application specific integrated circuit (ASIC). The processor 1210 may also be a microprocessor, a microcomputer, a processor chip, a controller, a microcontroller, a digital signal processor (DSP), a state machine, or a programmable logic device. The processor 1210 may also be a logical circuit, including a programmable gate array (PGA), such as a field programmable gate array (FPGA), or another type of circuit that includes discrete gate and/or transistor logic. The processor 1210 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 1200 further includes a main memory 1220 and a static memory 1230, where memories in the computer system 1200 communicate with each other and the processor 1210 via a bus 1208. Either or both of the main memory 1220 and the static memory 1230 may be considered representative examples of the memory 1051 of the controller 1050 in FIG. 10, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 1220 and the static memory 1230 are articles of manufacture and/or machine components. The main memory 1220 and the static memory 1230 are computer-readable mediums from which data and executable software instructions can be read by a computer (e.g., the processor 1210). Each of the main memory 1220 and the static memory 1230 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RANI memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 1200 further includes a video display unit 1250, such as a liquid crystal display (LCD), an organic light emitting diode (OLED), a flat panel display, a solid-state display, or a cathode ray tube (CRT), for example. Additionally, the computer system 1200 includes an input device 1260, such as a keyboard/virtual keyboard or touch-sensitive input screen or speech input with speech recognition, and a cursor control device 1270, such as a mouse or touch-sensitive input screen or pad. The computer system 1200 also optionally includes a disk drive unit 1280, a signal generation device 1290, such as a speaker or remote control, and/or a network interface device 1240.

In an embodiment, as depicted in FIG. 12, the disk drive unit 1280 includes a computer-readable medium 1282 in which one or more sets of software instructions 1284 (software) are embedded. The sets of software instructions 1284 are read from the computer-readable medium 1282 to be executed by the processor 1210. Further, the software instructions 1284, when executed by the processor 1210, perform one or more steps of the methods and processes as described herein. In an embodiment, the software instructions 1284 reside all or in part within the main memory 1220, the static memory 1230 and/or the processor 1210 during execution by the computer system 1200. Further, the computer-readable medium 1282 may include software instructions 1284 or receive and execute software instructions 1284 responsive to a propagated signal, so that a device connected to a network 1201 communicates voice, video or data over the network 1201. The software instructions 1284 may be transmitted or received over the network 1201 via the network interface device 1240.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment. A robot used to deploy the multiple stent delivery systems described herein may include a memory that stores instructions, a processor that executes the instructions, and one or more mechanical actuators to insert a catheter into a patient and retract outer sheaths to deploy stents. A robot may be guided by an operator, and may be provided with medical imaging used to assist the deployment of the stents as described herein.

Accordingly, multiple stent delivery system enables placement of two stents via a single catheter insertion. The capabilities described herein provide for situations in which stents are required on both left side and right sides of the vascular system and, for example, access can only be obtained on either the left side or the right side.

Although multiple stent delivery system has been described with reference to several exemplary embodiments, it is understood that the words that have been used are words of description and illustration, rather than words of limitation. Changes may be made within the purview of the appended claims, as presently stated and as amended, without departing from the scope and spirit of multiple stent delivery system in its aspects. For example, although two stents are shown in each embodiment described herein, more than two stents may be deployed by a multiple stent delivery system consistent with the descriptions herein. Although multiple stent delivery system has been described with reference to particular means, materials and embodiments, multiple stent delivery system is not intended to be limited to the particulars disclosed; rather multiple stent delivery system extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A stent delivery system, comprising:
a first outer sheath configured to surround a first stent; and
a second outer sheath configured to surround a second stent.

2. The stent delivery system of claim 1, wherein the stent delivery system is configured to deploy the first stent and the second stent by deploying the first stent, then retracting the first outer sheath while the second stent remains within the second outer sheath, then deploying the second stent, and then retracting the second outer sheath.

3. The stent delivery system of claim 1, wherein the first stent and the second stent are deployed via a single vascular access site.

4. The stent delivery system of claim 3, wherein the first stent and the second stent are deployed via a single catheter insertion.

5. The stent delivery system of claim 1, wherein the first stent is offset from the second stent along an axis of the second outer sheath before the first stent is deployed.

6. The stent delivery system of claim 1, further comprising:
a catheter that comprises the first stent, the first outer sheath, the second stent, and the second outer sheath, wherein the catheter is advanced to deploy the first stent and then retracted to deploy the second stent.

7. The stent delivery system of claim 1, further comprising:
an inner support shaft that runs through the first outer sheath and the second outer sheath.

8. The stent delivery system of claim 1, further comprising:
a drive system that separately retracts the first outer sheath and the second outer sheath.

9. The stent delivery system of claim 8, further comprising:
a handle that includes a first thumbwheel and a second thumbwheel,
wherein the drive system comprises the first thumbwheel and the second thumbwheel,
the first thumbwheel is configured to retract the first outer sheath, and
the second thumbwheel is configured to retract the second outer sheath.

10. The stent delivery system of claim 1, wherein the first stent is crimped over the second outer sheath.

11. The stent delivery system of claim 1, wherein the second outer sheath is disposed around the second stent and within the first stent prior to deployment such that a second diameter of the second outer sheath around the second stent is larger than a first diameter of the second outer sheath within the first stent.

12. The stent delivery system of claim 1, further comprising:
a third outer sheath aligned along an axis of the stent delivery system between the first outer sheath and a second outer sheath;
a first inner support shaft that runs through the first stent and the second stent; and
a second inner support shaft that runs through the first inner support shaft, wherein the second inner support shaft connects to a distal tip of the second outer sheath.

13. The stent delivery system of claim 12, wherein the stent delivery system is configured to deploy the first stent and the second stent by deploying the first stent, then retracting the first outer sheath while the second stent remains within the second outer sheath, then deploying the second stent while retracting the second outer sheath by advancing the second inner support shaft.

14. A catheter, comprising:
a first outer sheath configured to surround a first stent; and
a second outer sheath configured to surround a second stent, wherein the first stent is offset from the second stent along an axis of the catheter when the first stent and the second stent are assembled with the catheter.

15. The catheter of claim 14, further comprising:
a third outer sheath aligned along an axis of the catheter between the first outer sheath and a second outer sheath;
a first inner support shaft that runs through the first stent and the second stent; and
a second inner support shaft that runs through the first inner support shaft, wherein the second inner support shaft connects to a distal tip of the second outer sheath.

16. A method of deploying stents from a stent delivery system, comprising:
positioning the stent delivery system at a first disease site in an anatomical pathway; deploying a first stent from the stent delivery system at the first disease site;
retracting a first outer sheath from around the first stent while deploying the first stent from the stent delivery system;
positioning the stent delivery system at a second disease site in the anatomical pathway;
deploying a second stent from the stent delivery system at the second disease site; and
retracting a second outer sheath from around the second stent while deploying the second stent from the stent delivery system.

17. The method of deploying stents of claim 16, further comprising:
assembling the first stent, the first outer sheath, the second stent and the second outer sheath with the stent delivery system.
